(19) United States
(12) Patent Application Publication
(10) Pub. No.: US 2020/0101260 A1
(43) Pub. Date: Apr. 2, 2020

(54) Wellness Device and Method of Using Same

(71) Applicant: Benito Santoro, Princeton Junction, NJ (US)

(72) Inventor: Benito Santoro, Princeton Junction, NJ (US)

(21) Appl. No.: 16/590,340

(22) Filed: Oct. 1, 2019

Related U.S. Application Data
(60) Provisional application No. 62/739,840, filed on Oct. 1, 2018.
(60) Provisional application No. 62/740,412, filed on Oct. 2, 2018.
(60) Provisional application No. 62/742,264, filed on Oct. 5, 2018.
(60) Provisional application No. 62/753,061, filed on Oct. 30, 2018.

(51) Int. Cl. A61M 21/00; A61N 5/06; A61M 21/02; A61L 31/02

(52) U.S. Cl. CPC A61M 21/0094; A61N 5/0618; A61M 21/02; A61L 31/028; A61M 2021/0027; A61M 2205/6063; A61M 2205/0272; A61N 2005/0663; A61N 2005/0626; A61M 2205/3306; A61M 2021/005

(57) ABSTRACT

The invention helps treat the problem of our perception of our own holistic wellness, both physically, mentally and spiritually using accessibility, portability, form, senses and design to balance unbalanced thoughts and achieve more clear perception of ones self. Anchoring to this fundamental paradigm, using our wellness invention method, we create an experience that lends itself to restoring the user to a balanced state or to a specific desired outcome, or feeling. It achieves this by layering concepts (inclusive of theories, practices, ideas, beliefs) from several disciplines across all the senses. Here is presented a wellness device featuring an enclosed space suited to inducing a feeling of wellness in a patient feeling unbalanced.

### CROSS REFERENCE TO RELATED APPLICATIONS

The present application claims benefit under 35 U.S.C. § 119(e) to the following pending provisional patent applications: 62/739,840 filed on Oct. 1, 2018, 62/740,412 filed on Oct. 2, 2018, 62/742,264 filed on Oct. 5, 2018 and 62/753,061 filed on Oct. 30, 2018. The entirety of each of these provisional patent applications is hereby incorporated by reference.

### FIELD OF INVENTION

The invention is in the field of wellness. Particularly, the invention described is in the category of wellness enclosures. Also the invention is in subcategory of promoting wellness through isolation combined with stimuli including light and sound.

### BACKGROUND OF THE INVENTION

Kiosks that are self-directed with the purpose of improving health are well known; however, to date, the approach of these kiosks, apparatus, or devices has been to diagnosis disorders and dispense treatments. For examples see U.S. Patent Publication No. 2018/0158555 MEDICAL KIOSK AND METHOD OF USE, U.S. Patent Publication No. 2018/0181712 Systems and Methods for Patient-Provider Engagement, 2018/0052969 Method and Apparatus for Enhanced Personal Care, U.S. Patent Publication No. 2018/0048737 Method and Apparatus for Enhanced Personal Care with Linear Data Combining Attributes, U.S. Patent Publication No. 2014/0052458 TECHNIQUES FOR CUSTOMIZING AND DELIVERING WELLNESS PRODUCTS AND SERVICES, 20110191123 Virtual Lifestyle, Health, and Wellness Management System and Method, and U.S. Patent Publication No. 2008/0032267 Multimedia system and process for medical, safety, and health improvements. Others have promoted changing the environment to produce improvements in wellness, see U.S. Pat. No. 7,931,733 Method of producing hypoxic environments in occupied compartments with simultaneous removal of excessive carbon dioxide and humidity and U.S. Pat. No. 9,715,242 SYSTEMS, METHODS AND ARTICLES FOR ENHANCING WELLNESS ASSOCIATED WITH HABITABLE ENVIRONMENTS. Others have presented an apparatus for relaxation and psychosensorial stimulation see GB 2,201,599 “Relaxation and psychosensorial stimulation enclosure”, lacks the wellness-centered construction material and wellness-centered features such as objects and binaural sounds, therefore failing to optimally recenter the user. The invention as presented herein provides more effective and grounded means to recenter the patient.

The current paradigm of treatment in referenced devices attempt to provide feedback, plans, suggestions, actions, thoughts, “things” that a user will take with them to worry about or to simply concern oneself with, so it is not logical to imply an improved state of mental wellness when disengaging an apparatus that creates more things for the user to worry about afterward.

The patents/applications presented above are fundamentally different in the input or interaction and output or disengagement of the user. The references attempt to determine personal healthcare goals or concerns, and treat or assist with them in some way. And as an output or take-away, the systems provide readouts, treatment plans, advice, records, or things that the user has to address or be concerned about addressing or neglecting. The approach of the invention described herein, its method and the results of its use is the opposite. One may consider the mind does not want to hear about any “healthcare issues” and in turn the invention does not provide an output or any readout, which would only manifest “a thing” that didn't have to be real, to worry about. The invention provides a facility for the user where they disengage with less on their mind and no action items. This is diametrically in opposition of making people conscious and aware of their problems and then disengaging with awareness of their existing issues and additional new diagnostics, readouts, plans, advice, which becomes a thing to address.

While each of the aforementioned inventions may have their merits, all focus on the physical manifestation of human problems without regard to the spiritual and mental state of a patient, there is a need for an apparatus that improves the wellness of a patient. Such a wellness apparatus for improving one's state of wellness, whether physical, mental, or spiritual, real, perceived or imagined or in any conceivable way in which a person can feel better. Here is presented a wellness device that allows a patient can achieve wellness through the use of the device without engaging a clinician.

### BRIEF SUMMARY OF THE INVENTION

Here it is presented a wellness apparatus for improving a patient's state of wellness, comprising: An enclosure and a wellness feature.

Said enclosure having an interior and an exterior. Said enclosure is constructed of a series of at least four panels.

The first panel has an inside and an outside. The first panel also has a left side and right from the perspective of viewing the inside face of the panel. It also has a top side and a bottom side, and being substantially flat in shape.

The enclosure is also constructed of two side panels. Each side panel has an inside and an outside. Also, each side panel has a left side and right side from the perspective of viewing the inside face of the panel. Also, each panel has a top side and a bottom side; each being substantially flat in shape. Furthermore, one side panel is removably connected at its right side to the left side of the first panel and the other panel is removably connected at its left side to the right side of the first panel.

The enclosure is also constructed of a rear panel. The rear panel has an inside and an outside. Also, the rear panel has a left side and right side from the perspective of viewing the inside face of the panel. Also, the rear panel has a top side and a bottom side. The rear panel is substantially flat in shape. Furthermore, the rear panel's left side is removably connected to a side panel and the right side of the rear panel is removably connected to a side panel. The rear panel is further constructed with door portion that is connected by a hinge allowing for the opening and closing of said door portion.

Optionally, the enclosure may include a roof. Such a roof has a top side and bottom side and four side edges. Furthermore, each side edge is removably connected to the top of each panel such that the top side of the roof is on the exterior of the enclosure and the bottom side of the roof faces the interior of the enclosure;

Optionally, the enclosure may include a floor. Such a floor has a top side and bottom side and four side edges. Furthermore, each side edge is removably connected to the bottom of each panel such that the bottom side of the floor is on the exterior of the enclosure and the top side of the floor faces the interior of the enclosure.

Affixed to the first panel, is a display screen electronically coupled to a control processor.

At least one lighting unit is affixed to the interior of the enclosure. The lighting unit is capable of illuminating the interior in all the colors of the visual spectrum, as well as in combinations thereof. Said lighting until is electronically coupled to the control processor.

In most embodiments, at least one speaker is affixed to the interior of the enclosure with headphones and an AC jack available. The speaker is capable of creating sound within the enclosure. The speaker is electronically coupled to the control processor.

The control processor is configured to direct at least one lighting unit to produce a steady or modulating light to enhance the wellness of a human. Also, the control processor is configured to direct at least one speaker unit to produce sounds to enhance the wellness of a human.

The wellness feature may be any of the wellness features described in the wellness feature section.

### BRIEF DESCRIPTION OF FIGURES

FIG. 1 depicts a flow chart of activities during operation of the invention described herein in an exemplar embodiment.

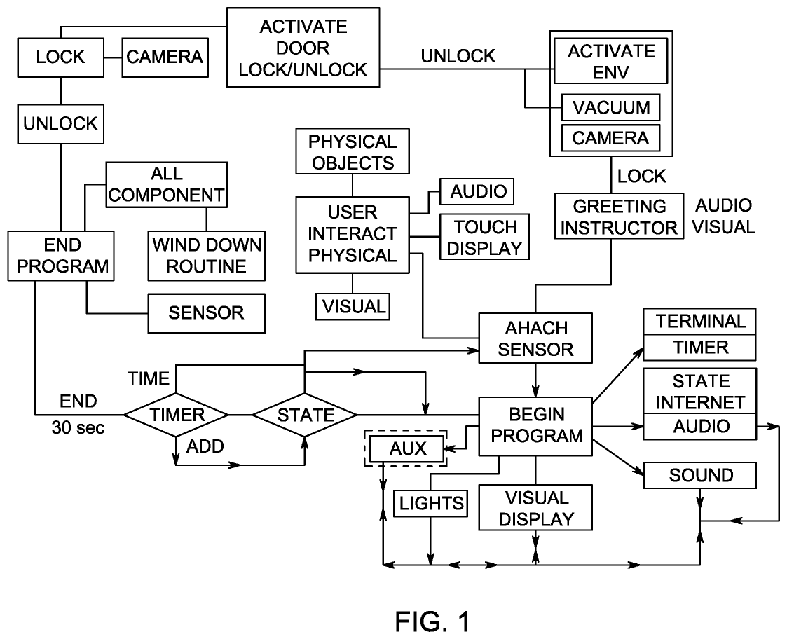

FIG. 2 depicts the electrical connectivity of components of the invention described herein in an exemplar embodiment.

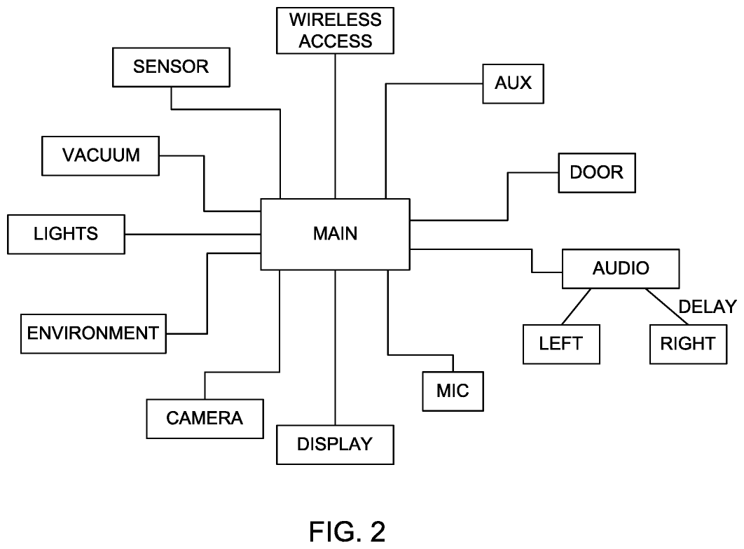

FIG. 3 depicts an exterior view of an exemplar embodiment of the invention described herein.

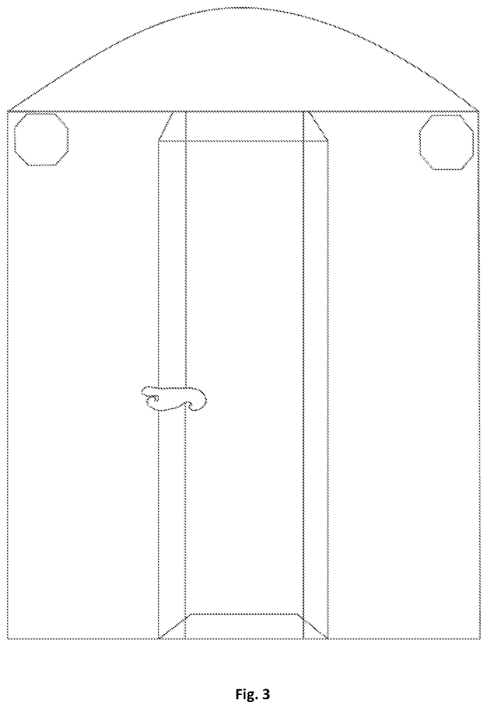

FIG. 4 depicts an interior view of an exemplar embodiment of the invention described herein.

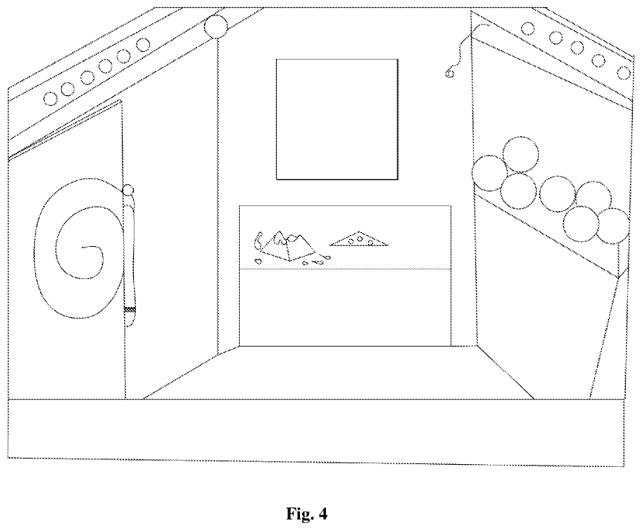

### DETAILED DESCRIPTION OF THE INVENTION

### Objective and Advantages

The invention present herein is designed to facilitate the users' ability to disassociate with thoughts and feelings, in other words, leaving one with less on their mind and lighting their feeling of responsibility and thereby improving one's perceived or real state of being.

A process is provided to allow a user or patient to achieve a greater state of wellness using the apparatus and the components provided described herein.

### Overview of the Invention

Here it is presented a wellness apparatus for improving a patient's state of wellness, comprising: An enclosure and a wellness feature. The apparatus and its components provide an environment to treat the problem of our perception of our own holistic wellness, both physically, mentally and spiritually. The apparatus treats the problem of our perception of our own holistic wellness, both physically, mentally and spiritually using accessibility, portability, form, senses and design to balance unbalanced thoughts and achieve more clear perception of one's self. Anchoring to this fundamental paradigm, using our wellness invention method, we create an experience that lends itself to restoring the user to a balanced state or to a specific desired outcome, or feeling. It achieves this by layering concepts (inclusive of theories, practices, ideas, beliefs) from several disciplines across all the senses. Here is presented a wellness device featuring an enclosed space suited to inducing a feeling of wellness in a patient feeling unbalanced.

### Wellness Feature

As used herein a “wellness feature” means one of the following enhancers of wellness.

At least one crystalline sphere or crystalline spheroid. Preferred crystals are listed and described in the crystal section provided herein.

At least one device with movable parts designed to promote wellness; at least one such object is the subject of a co-pending patent application by the inventor of the instant application.

Or at least one sound or light regime.

### Construction Materials

It should be apparent to anyone of skill in the art that the invention as described herein can be constructed with any modern engineering means. While the practitioner of the invention may choose any reasonable construction material to create the panels of the invention described, preferred materials include salt, stone, rock, crystals, gemstones, glass, mud, minerals and combinations thereof. More preferably the panels of the enclosure are constructed from obsidian, calcite, lodestone or any natural rock or glass or any lava rock, quartz, crystals, gemstones, minerals, salt or combinations thereof.

In general terms the enclosure of the inventions ought to be constructed predominately from naturally occurring minerals, rocks, stones, gemstones, crystals, salt, lava rocks and glass, with particular focus on healing stones that naturally occur such as quartz, obsidian, salt, gemstones, crystals, lava rock and rock glass where the aforementioned list is the material predominantly exposed to the user and their experience.

It is understood that such the above-mentioned materials have a common physical feature in that each emits background radiation of a specific and measurable quantity. Particularly, the preferred construction materials emit radiation in the form of 40K of at least 1 Bq per Kg. In a more preferred, embodiment construction materials emit radiation in the form of ⁴⁰K of at least 5 Bq per Kg. In a still more preferred embodiment, construction materials emit radiation in the form of ⁴⁰K of at least 1 Bq per Kg. Further descriptions of the radio-signatures of preferred constructions materials can be found in, inter alia, Aquino and Pecequilo, 2011 International Nuclear Atlantic Conference—IN AC 2011 Belo Horizonte, MG, Brazil, Oct. 24-28, 2011 Associargo Brasileira De Energia Nuclear—Aben ISBN: 978-85-99141-04-5, hereby incorporated by reference in its entirety.

### Additional Components

The invention may also provide a circulation system preferably in the form of an HVAC system, The apparatus will contain a circulating fan, specifically designed for optimal airflow, creating a vortex of air that circulates up from the base, while the fan spins it creates a light electrical charge “static electric” and this will additionally help cleanse the energy centers, simulating a natural vortex, but also utilizing electricity as a means of improved wellness.

The invention may also comprise have a motor, preferably an alternating current motor, Such motor is preferred to have the ability to convert to wireless charging capabilities. This would allow the apparatus to operate in locations not easily serviced by the electrical grid. It is understood that installing solar panels on the roof or similar self-powering means will allow the invention it be used in almost any location.

It is understood the CPU of the invention can be configured to accept some form of payment or recognize that a payment has been provided before providing access to the enclosure in the apparatus. In such embodiments, the apparatus is constructed with a door having a locking means. The unit may be constructed with a payment receiving unit configured to receive cash, coin, credit card or other payment means including electronic payment. Such payment and locking systems are well known to those skilled in the art.

### Crystals

Certain crystals can be used with the device to enhance wellness. Such crystals can be used as described here; however, the use of crystals is not limited to the specific crystal described.

List of Chakra Stones

Each chakra is typically associated with one or several stones. Here's a list to help you chose your healing crystal depending on the energy center which to the subject of focus.
Root chakra stones: Bloodstone, Tiger's Eye, Hematite, Fire Agate, Black Tourmaline
Sacral chakra stones: Citrine, Carnelian, Moonstone, Coral
Solar Plexus chakra stones: Malachite, Calcite, Citrine, Topaz
Heart chakra stones: Rose Quartz, Jade, Green Calcite, Green Tourmaline
Throat chakra stones: Lapis Lazuli, Turquoise, Aquamarine
Third eye chakra stones: Amethyst, Purple Fluorite, Black Obsidian
Crown chakra stones: Selenite, Clear Quartz, Amethyst, Diamond

In a preferred embodiment, the crystal wellness-promoting devices can be embedded with a magnetic substance. In a more preferred embodiment, the magnetic substance is naturally occurring. In a still more preferred embodiment, the magnetic substance is lodestone.

### Crystal and Sound Relationship

Attuning to the natural vibrations has been understood since ancient times. All the sounds the invention uses are tuned to 432 Hz scale as described herein. Such sounds can be used with the invention in conjunction with generally understood chakra methodology utilizing both color and sounds associated with each energy vortex or nerve ganglia branching out from the spinal column. It is believed these tones create harmonic resonance for each energy center or nerve ganglia, this attunes it to its natural frequency, creating a feeling of refreshing it, like refreshing a frozen web page. In addition to cycling through these frequencies, these frequencies may be played as a binaural beat, between 4-6 Hz in the difference between left and right speakers, producing this delay also further serves to refresh the nerves and energy center in your body including your brain waves.

The following frequencies may be used:

Crown Chakra
Color: Violet/Purple
Frequency: 480 Hz, 768, 963

Opening this chakra will help tap into a deep spiritual understanding. Gemstones that will aid the Crown chakra include diamond and clear quartz.

Third Eye Chakra
Color: Indigo
Frequency: 432 Hz, 720, 852

This chakra is our link to the universal unconsciousness. Opening this chakra can aid in clear sight, intuition, integrity, and lessens feelings of despair. Gemstones amethyst, moonstone and augilite.

Throat Chakra
Color: Blue
Frequency: 384 Hz, 672, 741

Opening the Throat chakra improves clairaudience. Gemstones that will aid the Throat chakra include turquoise, aquamarine, lapis, sodalite, quartz, angelite, aqua aura.

Heart Chakra:
Color: Green
Frequency: 341-364 hz, 552-624,639

Opening the Heart Chakra allows a person to love more, empathize, and feel compassion. Gemstones that will aid the Heart chakra include emerald, tourmaline, aventurine, malachite, rose quartz, and rhodonite

Solar Plexus Chakra
Color: Yellow
Frequency: 324 Hz, 528, 528

When this chakra is open, it acts to empower a person and help them find their personal strengths. It will help turn dreams and goals into reality. Gemstones that will aid the Solar Plexus chakra include amber, topaz, citrine, yellow calcite, yellow jasper.

Sacral Chakra
Color: Orange
Frequency: 288 Hz, 480, 417

Opening this chakra will free fertility and inherent creativity. The Sacral chakra stimulates sexuality and emotions. Gemstones that will aid the Sacral chakra include carnelian, coral, orange jasper, orange jade.

Root Chakra
Color: Red
Frequency: 256 Hz, 432, 396

Groundedness, trust, belonging, lessens feelings of mistrust. Gemstones that will aid the Root chakra include lodestone, ruby, garnet, smokey quartz, obsidian, hematite and onyx.

### Fan

The invention may further provide a fan unit. It is preferred that such a fan unit is capable of producing a vortex in the interior of the apparatus. It is still more preferred that the vortex can be salted with charged particles, including ions, electrons and protons. The vortex can be coupled with any device capable of charging particle and/or providing particles in a charged state.

### Retinal Scanner

The invention may further provide a retinal scanning unit. It is preferred that such a retinal scanner is capable of electronically transmitting an optic scan of a retina to the central processor. The central processor is configured by software and/or hardware means to analyze the transmitted optic scan. In a preferred embodiment, the central processor is capable analyzing the well-being of a patient by means of the optic scan. In equally preferred embodiment the central processor is capable of recognizing a patient by analyzing the optic scan.

### Sounds

The invention is capable of producing sounds. Such sounds can be created by many known means. Such means can achieved through the use of audio speakers, the use of headphones and or tuning forks. Audio speakers are dynamic and can be tuned to any perceptible frequency. The dynamic quality is also a facet of headphones. Tuning forks, by contrast, are constructed to emit a single frequency when activated. Tuning forks can be constructed to emit any perceptible frequency. It is preferred that the tuning forks used with the invention are constructed to emit preferred sound frequencies as described herein.

Sounds can be used with the device to enhance wellness, such sounds can be used as described in this section, the sounds capable of being used with the device are not limited to the specific sounds described. One skilled in the art will recognize the genre of sounds that can be used with the described invention based on the species described.

Sounds, music that can be used with the device are the tones from the musical scale based on 432 hertz, which produces tones which are in harmony with nature and also our own bodies as well as the universe, specifically hertz of 396, 417, 432, 528, 639, 741, 852, 963, 174, 285 963 and 144. This can scale down to 1 and 2 hz using golden ratio, approximately 1.68133

Also using slightly delayed signals on one speaker compared to another, left and right side, creating a slight delay creates another opportunity to improve one's wellness, by refreshing our mental wavelengths, creating a calmer mind, a feeling of improved wellness. These sounds are referred to as “Binarual Beats”. Such sounds can be 1 Hz up to 50 Hz or more difference, it is preferred if the delays are in the 2 to 34 Hz range in terms of difference in lag, as to produce that pulsing sound, that is the Hz that is heard. Using binaural beats (delay left is right) at around 4 Hz difference. This helps the mind slow down as it has been shown the brain resonates around 4 Hz when in deep meditation. This with the described invention to improves one's mental wellness after using the invention as compared to their mental state prior to using it.

Sounds can be used in combination with the presence of crystal to enhance the wellness effect of the device. The following are the core tones that align with the chakras. The crystals also will follow the nature of the crystals and gens associated with each chakra:

The main six Solfeggio frequencies are:
396 Hz—Liberating Guilt and Fear
417 Hz—Undoing Situations and Facilitating Change
528 Hz—Transformation and Miracles
639 Hz—Connecting/Relationships
741 Hz—Expression/Solutions
852 Hz—Returning to Spiritual Order

### Internal Environment

The internal environment is preferably controlled using A/C/Heater or other methods or any device that can modify the environment (including fans) that creates vacuum as well as maintains passively the environment. The environment may also be controlled by the space the device is located, i.e. the ambient environment in which the enclosure is located.

### Light Based Chromotherapy or Photobiology

Chromotherapy is the science of using colors to adjust the body and promote wellness. Each color possesses frequencies of a specific hertz, and each frequency is related to the wellness of a particular party of the body.

A Brief Overview of Colors Used in Chromotherapy and their Benefit:

Red: Red is believed to increase the pulse, raise blood pressure and increase the rate of breathing. Red would be applied to support circulatory and nervous functions.

Strong Pink: Strong pink acts as a cleanser, strengthening veins and arteries.

Pink: Pink activates and eliminates impurities in the bloodstream.

Orange: Orange is a mixture of red and yellow. Activates and eliminates localized fat. Assists with asthma and bronchitis

Strong Yellow: Strong yellow strengthens the body and activates internal tissues.

Yellow: Yellow, the brightest color used in chromotherapy, has been used to purify the skin, help with indigestion, strengthen the nervous system, treat glandular diseases, hepatitis and lymphatic disorders and assist metabolism.

Green: Green, a color associated with harmony, provides a neutral, positive calming effect.

Strong Green: Strong green provides anti-infectious, anti-septic and regenerative stimulation.

Strong Blue: Strong blue lubricates joints, helps address stress, nervous tension and infections.

Blue: Blue promotes relaxation and calm. Blue exhibits tranquilizing qualities often used to relieve headaches and migraines, colds, stress, nervous tension, rheumatism, stomach pains, muscle cramps and liver disorders. Blue is thought to have a positive effect on all kinds of pain.

Indigo: Indigo is used to address conditions involving the eyes, ears and nose. It has a calming, sedative effect.

Violet: Violet is used to calm the nervous system, soothe organs and relax muscles. Violet has meditative qualities and is often used to treat conditions of the lymphatic system and spleen, as well as urinary disorders and psychosis.

### Process

The invention can be configured to implement a system is referred to as the 3-6-9 path to better mental wellness, more information can be found in [a yet unpublished manuscript authored by the inventor]. The method or system works with the use of vivid, sensory stimulating, attention-grabbing statements, to capture the user's imagination and attention, clearing out their worries or thoughts, the user will feel less burdened, the feelings will go away and the user's general outlook will be improved. When the user ends their interaction, their mental wellness will be improved.

While not limited to the specifics disclosed in the following, the invention can be practiced in this manner:
3 cycles (6 minutes)
9-7 chakras, 2 exercises to feel (physical (“real” physical interaction and a mental “imagined or recall”)
See+Hear=Feel
Touch=Feel
Taste+Smell

Step of the Process
(1) Brief introduction followed by the dimming and calming of the environment and music begins
(2) User is prompted to think about or say what is troubling them, must feel it to make it real.
(3) Sequences through lights and sounds in a progression through the chakras while the user interacts with objects
(4) Prompt user to imagine (in vivid feeling detail) an amazing experience, something they would want to do again. See it in your mind, hear it in your mind.
(5) Sequences through lights and sounds in a progression through the chakras while the user interacts with objects
(6) Prompts the user to perform a passive physical exercise this time, like relax your “neck”
(7) Sequences through lights and sounds in a progression through the chakras while the user interacts with objects
(8) Provides the user with a vivid thought meant to stimulate their imagination and focus on self, and not begin to worry about others.

I claim:

1. A wellness apparatus for improving a patient's state of wellness, comprising:
a) An enclosure with an interior and an exterior which is constructed of
a first panel having an inside and an outside, also having a left side and right from the perspective of viewing the inside face of the panel, also having a top side and a bottom side, and being substantially flat in shape;
the enclosure is also constructed of two side panels each having an inside and an outside, also each having a left side and right side from the perspective of viewing the inside face of the panel, also having a top side and a bottom side; each being substantially flat in shape, wherein one side panel is removably connected at its right side to the left side of the first panel and the other panel is removably connected at its left side to the right side of the first panel;
the enclosure is also constructed of a rear panel having an inside and an outside, also having a left side and right side from the perspective of viewing the inside face of the panel, also having a top side and a bottom side, and being substantially flat in shape; wherein the left side is removably connected to a side panel and the right side is removably connected to a side panel; the rear panel is further constructed with door portion that is connected by a hinge allowing for the opening and closing of said door portion;
Optionally the enclosure may include a roof with a top side and bottom side and four side edges, wherein each side edge is removably connected to the top of each panel such the top side of the roof is on the exterior of the enclosure and the bottom side of the roof faces the interior of the enclosure;
Optionally the enclosure may include a floor with a top side and bottom side and four side edges, wherein each side edge is removably connected to the bottom of each panel such the bottom side of the floor is on the exterior of the enclosure and the top side of the floor faces the interior of the enclosure;
Affixed to the first panel, is a display screen electronically coupled to a control processor;
At least one lighting unit is affixed to the interior of the enclosure; said lighting unit is capable of illuminating the interior in all colors of the visual spectrum and in combinations thereof; said lighting until is electronically coupled to the control processor;
A sound generating device is affixed to at the interior side of at least one panel, said sound generating device is are capable of producing wellness sounds and is coupled to the control processor;
The control processor is configured to direct at least one lighting unit to produce a steady or modulating light to enhance the wellness of a human; the control processor is configured to direct at least one speaker unit to produce sounds to enhance the wellness of a human;
Wherein at least one of the first panel, side panels and rear panel is constructed from a material that shows activity of ⁴⁰K of at least 5 Bq/Kg
b) at least one wellness-promoting feature.

2. The wellness apparatus of claim 1, wherein the wellness-promoting feature is a wellness-promoting object with movable parts positioned within the enclosure.

3. The wellness apparatus of claim 1, wherein the wellness-promoting feature is a crystalline wellness-promoting object positioned within the enclosure.

4. The wellness apparatus of claim 1, wherein the wellness-promoting feature is a light pattern or program that provides chromotherapy.

5. The wellness apparatus of claim 1, wherein the sound generating device is at least two speakers.

6. The wellness apparatus of claim 1, wherein the sound generating device is a pair of headphones electronically wherein the electronic coupling to the central processor passes through an audio jack.

7. The wellness apparatus of claim 1, wherein the sound generating device is a tuning fork mechanically coupled to an activating hammer which is electronically coupled to the central processor.

8. The wellness apparatus of any of claims 5-7, wherein the wellness sound produced are configured to be a binaural beat.

9. The wellness apparatus of any of claims 5-7, wherein the wellness sounds are tuned to a scale centered on 432 Hz.

10. The wellness apparatus of any of claims 1-9, wherein at least one panel interior face is constructed substantially from naturally occurring rock.

11. The wellness apparatus of claim 10, wherein the naturally occurring rock is obsidian lodestone or calcite.

12. The wellness apparatus of any of claims 1-9, wherein at least one panel interior face is constructed substantially from naturally occurring crystal.

13. The wellness apparatus of claim 12, wherein the naturally occurring crystal is in the quartz family.

14. The wellness apparatus of any of claims 1-9, wherein at least one panel interior face is constructed substantially from gemstones.

15. The wellness apparatus of claim 14, wherein gemstone is naturally occurring and mined.

16. The wellness apparatus of claim 14, wherein gemstone is synthesized.

17. The wellness apparatus of any of claims 1-9, wherein at least one panel interior face is constructed substantially salt.

18. The wellness apparatus of any of claims 1-9, wherein at least one panel interior face is constructed substantially from wood.

19. The wellness apparatus of any of claims 1-9, wherein at least one panel interior face is constructed substantially from marble.

20. The wellness apparatus of any of claims 1-9, wherein at least one panel interior face is constructed substantially from wood.

21. The wellness apparatus of any of claims 1-7, wherein at least one panel interior face is constructed substantially from metal.

22. The wellness apparatus of claim 19, wherein the metal is steel, iron, aluminum or an alloy thereof.

23. The wellness apparatus of any of claims 1-9, wherein at least one panel interior face is constructed substantially from a glassy rock.

24. The wellness apparatus of any of claims 1-9, further comprises at least one microphone positioned in the interior of the enclosure, optionally the microphone is electronically connected to a computer capable of voice recognition.

25. The wellness apparatus of any of claims 1-9, wherein the interior lighting color and intensity is controlled by the central processor running a program that varies the color and intensity of the lights over time to create a feeling of wellness in the patient.

26. The wellness apparatus of claim 2, wherein the wellness-promoting object with moving parts is sanitized by a UV light positioned in the enclosure.

27. The wellness apparatus of claim 3, wherein the crystalline wellness-promoting object is sanitized by a salt bath positioned in the enclosure.

28. The wellness apparatus of any of claims 1-9, further comprising at least one camera positioned in the interior of the enclosure, optionally the camera on/off status is controlled by the opening and closing of the door positioned within the rear panel.

29. The wellness apparatus of claim 28, wherein the camera is electronically connected to a computer capable of facial recognition

30. The wellness apparatus of any of claims 1-9, further comprising a fan that is capable of creating a vortex.

31. The wellness apparatus of claim 30, wherein the vortex is salted with charged particles.

32. The wellness apparatus of claim 3, wherein the crystal is embedded with a magnetic substance.

33. The wellness apparatus of claim 3, wherein magnetic substance is lodestone.

34. The wellness apparatus of any of claims 1-9, wherein the apparatus further comprises a retinal scanner.

35. The wellness apparatus of any one of claims 1-12, wherein the enclosure is simulated by a virtual reality machine.

36. A process for promoting wellness in a patient, using an apparatus comprising
an enclosure with an interior and an exterior which is constructed of
a first panel having an inside and an outside, also having a left side and right from the perspective of viewing the inside face of the panel, also having a top side and a bottom side, and being substantially flat in shape;
the enclosure is also constructed of two side panels each having an inside and an outside, also each having a left side and right side from the perspective of viewing the inside face of the panel, also having a top side and a bottom side; each being substantially flat in shape, wherein one side panel is removably connected at its right side to the left side of the first panel and the other panel is removably connected at its left side to the right side of the first panel;
the enclosure is also constructed of a rear panel having an inside and an outside, also having a left side and right side from the perspective of viewing the inside face of the panel, also having a top side and a bottom side, and being substantially flat in shape; wherein the left side is removably connected to a side panel and the right side is removably connected to a side panel; the rear panel is further constructed with door portion that is connected by a hinge allowing for the opening and closing of said door portion;
Optionally the enclosure may include a roof with a top side and bottom side and four side edges, wherein each side edge is removably connected to the top of each panel such the top side of the roof is on the exterior of the enclosure and the bottom side of the roof faces the interior of the enclosure;
Optionally the enclosure may include a floor with a top side and bottom side and four side edges, wherein each side edge is removably connected to the bottom of each panel such the bottom side of the floor is on the exterior of the enclosure and the top side of the floor faces the interior of the enclosure;
Affixed to the first panel, is a display screen electronically coupled to a control processor;
At least one lighting unit is affixed to the interior of the enclosure; said lighting unit is capable of illuminating the interior in all colors of the visual spectrum and in combinations thereof; said lighting until is electronically coupled to the control processor;
A sound generating device is affixed to at the interior side of at least one panel, said sound generating device is are capable of producing wellness sounds and is coupled to the control processor;
The control processor is configured to direct at least one lighting unit to produce a steady or modulating light to enhance the wellness of a human; the control processor is configured to direct at least one speaker unit to produce sounds to enhance the wellness of a human;
Wherein at least one of the first panel, side panels and the rear panel is constructed from a material that shows activity of ⁴⁰K of at least 5 Bq/Kg and
at least one wellness-promoting feature.
Wherein, the following steps are performed and control by the central processor:
(1) the light emitted at least the lighting unit is dim and sound is emitted from at least one speaker;
(2) At least one speaker emits the sound of a voice prompting the patient to think about or say what is troubling them;
(3) Sequences through lights and sounds in a progression through the described chakras while the patient is prompted by the speaker to interact with objects;
(4) At least one speaker prompts the patient to imagine an experience;
(5) The lighting unit and speakers progress through the described chakras the patient is prompted by at least one speaker to interact with objects,
(6) At least one speaker prompts the user to perform a passive physical exercise;
(7) The lighting unit and speakers progress through the described chakras the patient is prompted by at least one speaker to interact with objects,
(8) At least one speaker provides the patient with a vivid thought.

37. The process of claim 36, wherein the sound generating device is at least two speakers.

38. The process of claim 36, wherein the sound generating device is a pair of headphones electronically wherein the electronic coupling to the central processor passes through an audio jack.

39. The process of claim 36, wherein the sound generating device is a tuning fork mechanically coupled to an activating hammer which is electronically coupled to the central processor.
